(19) United States
(12) Patent Application Publication
(10) Pub. No.: US 2008/0283058 A1
(43) Pub. Date: Nov. 20, 2008

(54) Inhaler

(75) Inventors: Andrew John Ede, Cambridge (GB); Daniel Peterson Godfrey, Cambridge (GB)

Correspondence Address: RENNER OTTO BOISSELLE & SKLAR, LLP, 1621 EUCLID AVENUE, NINETEENTH FLOOR, CLEVELAND, OH, 44115, US

(73) Assignee: EG TECHNOLOGY LIMITED, Cambridge (GB)

(21) Appl. No.: 10/597,271

(22) PCT Filed: Jan. 21, 2005
(86) PCT No.: PCT/GB2005/000159

§ 371 Date: Aug. 5, 2008

(30) Foreign Application Priority Data
Jan. 23, 2004 (GB) 0401419.7

(51) Int. Cl. A61M 16/00; A61M 15/00

(52) U.S. Cl. 128/203.21; 128/203.15

(57) ABSTRACT

The invention provides an inhaler of the non-reservoir kind (i.e. one which operates by dispensing individually packaged doses) characterised by the feature that a moisture-proof barrier—which may optionally comprise substantially or partly an external region of the inhaler-encloses the dose-storage region of the inhaler without enclosing the doses individually, and incorporates a similarly moisture-proof aperture through which the medicament doses can, in use, be individually dispensed. The invention also provides a method of filling such an inhaler.

### FIELD OF THE INVENTION

The invention relates to apparatus for storing and dispensing medicaments intended to be administered by inhalation. It relates, in particular, to inhalers of the non-reservoir kind. The invention also relates to a method of filling such an inhaler.

### PRIOR ART KNOWN TO THE APPLICANT

Devices that store and dispense medicaments intended for administration by inhalation are known in themselves. These devices, known generically as ‘inhalers’ are used to store and deliver pharmaceutical preparations, by inhalation, for a number of diseases. Most commonly, inhalers are used to administer bronchodilators for the treatment of diseases such as asthma. The pulmonary route for administration of medicament also offers advantages for the delivery of other drugs such as those used to treat allergic rhinitis. Increasingly, the pulmonary route is also being used for the delivery of drugs and other agents for systemic therapy, including, for example, insulin for treatment of diabetes.

The delivery mechanism relies on creating either a dispersion of solid particles or liquid droplets in a gas phase (usually air) to form an aerosol that may be inhaled by a patient. Alternatively, the medicament can be in the form of a vapour. In the case of aerosols, the particle or droplet size distribution of the dispersed phase is crucial to ensuring the delivery of the medicament to the correct point in the respiratory tract.

In order to ensure that the medicament is delivered in the correct way, and also at the correct dose, it is essential, therefore, to protect both the physical and chemical integrity of the medicament whilst it is stored in the delivery apparatus. Typically, these devices are used both frequently, and sporadically by a user. The devices must, therefore, be capable of maintaining the medicament preparation in good order over an extended period of time. Many medicaments are prone to degradation when exposed to either moisture or oxygen. Dry-powder formulations are also susceptible to physical degradation by the effects of moisture and other agents, which can disrupt the ability of the powder to be dispersed as an aerosol with a predetermined particle size distribution. Thus, inhaler technology has developed to afford such protection.

There are effectively two categories of inhaler devices: the ‘reservoir-type’ in which the medicament is stored within the inhaler in a single reservoir, and a ‘non-reservoir type’ in which the medicament is stored within the inhaler as a number of individual, pre-determined doses.

International patent application PCT/EP02/11311 (published as WO03/035151) is a typical example of the ‘non-reservoir’ type of inhaler. In this device, individual doses are stored in a medicament carrier comprising a flexible strip defining a plurality of pockets, each of which contains a dose of medicament that can be inhaled. The strip comprises a base sheet in which blisters are formed to define the pockets and a lid sheet, hermetically sealed to the base sheet in such a manner that the lid sheet and the base sheet can be peeled apart.

In inhalers of this type, these individual pockets, often formed of a plastics or metallic material, provide the protection against the degredative effects of moisture, oxygen and other agents. The sealed area between the base and lid sheets is often more permeable than the individual materials themselves, either due to the material properties of the adhesives used, or to the material properties of the heat-sealed unit. As a result, the distance between individual pockets and between the pockets and the edge of the strip needs to be substantially larger than the thickness of either the base sheet or lid sheet. Thus, the overall size of the medicament carrier is significantly larger than the volume of the medicament that it contains. Consequently, the number of doses that may be contained within an inhaler unit that is a convenient size is limited and less than might be desirable.

International patent application PCT/GB00/02017 (published as WO00/74754) illustrates an example of a ‘reservoir-type’ inhaler. In inhalers of this type, the medicament, which may be in the form of a dry powder, is stored in a single reservoir, and metering of the individual doses is carried out by a mechanism incorporated in the inhaler itself. Inhalers of this type have the advantage that a large amount of medicament may be stored within the single reservoir but have the drawback of either having to provide a very sophisticated dosing mechanism to ensure consistency of individual doses, or the delivery of doses of inconsistent size. In the example of such a ‘reservoir-type’ inhaler described in PCT/GB00/02017, the medicament is protected during storage in the single reservoir by manufacturing the reservoir and its lid of a material that constitutes a moisture proof barrier. It is also necessary in this type of inhaler to provide a moisture resistant sealing means as part of the dosing mechanism to prevent ingress of moisture into the main reservoir chamber.

UK patent GB 2 016 735 describes a “non reservoir-type” inhaler for containing one or more pre-dosed capsules of powdered medicament. The medicament is sealed within these capsules, and the inhaler provides a mechanism to release medicament from its protective capsule into a position where it can be entrained in an airflow created by a user sucking on a mouthpiece; the sucking action draws air into the device through apertures provided in the inhaler's casing. In some embodiments of this device, a lid is provided, the opening of which actuates the release of the medicament, and which, in its closed position, covers the mouthpiece. Whilst the lid and the outer casing provide a degree of protection to the capsules, and may prevent foreign bodies entering the device, the provision of apertures within the walls, the fact that the lid is optional, and the lack of any material specification result in the device being far from moisture-proof, moisture-sensitive medicament must, therefore be protected by the capsules themselves. The positive provision of apertures in the walls of the device, and the optional lid has the result that air outside the casing can freely exchange with the air inside the device with the consequence that the device provides no control over the environment surrounding the individual doses.

Many reservoir-type devices are manufactured from injection-moulded plastics components. The plastics used in these components inherently have a relatively high moisture and oxygen permeability. In these devices the drug is relatively unprotected from degradation and therefore these devices are only suitable for the more stable drugs. Some designs attempt to reduce this effect by placing a desiccant in the reservoir, however the efficacy of this approach is limited as the desiccant can become saturated and it is sometimes considered undesirable to have a non-pharmaceutical ingredient (the desiccant) in direct communication with the pharmaceutical active.

It is an object of the present invention to provide an inhaler that is both able to protect the medicament stored within it from the effects of moisture, oxygen and other degredative agents as well as allowing the storage of an increased number of individual pre-determined doses of medicament within the constraints of a device that is convenient for a user.

### SUMMARY OF THE INVENTION

The invention takes as its starting point the last-named specification, GD 2 016 735, as being of the “non-reservoir” type, and having an outer, albeit not moisture-proof, casing.

The invention provides an inhaler of the non-reservoir kind (i.e. one which operates by dispensing individually packaged doses) characterised by the feature that a moisture-proof barrier—which may optionally comprise substantially or partly an external region of the inhaler—encloses the dose-storage region of the inhaler without enclosing the doses individually, and incorporates a similarly moisture-proof aperture through which the medicament doses can, in use, be individually dispensed.

In the broadest aspect of the invention, it is to be understood that the term “moisture-proof barrier” means a barrier whose combination of material properties and construction is such as to resist the passage of water—either in liquid or vapour form—into the dose-storage region of the inhaler from the outside. Suitable materials would include plastics, metals and glasses (or composites thereof).

Some medicaments, however, are particularly sensitive to moisture, and for inhalers capable of storing these medicaments for extended periods, the “moisture-proof barrier” needs to provide a greater defence against the ingress of water, again either in liquid or vapour form. Suitable materials for applications such as this would include specialist plastics with particularly high moisture resistance, metals and glasses (or composites thereof). Current plastics capable of providing a suitable barrier would include the COC (cycloolefin copolymer) family of materials, PVdC (polyvinylidene chloride) and PCTFE (polychlorotrifluoroethylene). Metals suitable for the purpose include aluminium, stainless steel, silver, gold and copper. Generically, such materials may be referred to as “high barrier” materials.

To determine the suitability of any material/construction combination, the skilled addressee may perform routine tests to determine the water penetration rate. For such sensitive applications, it is the penetration of water vapour that it likely to be critical. The moisture-sensitivity of medicament preparations is likely to vary between medicaments and the various ways of preparing them for delivery by inhalation. However, as a guide, and for typical moisture-sensitive applications, the “moisture-proof barrier” should allow a moisture penetration of no more than 0.1 mg of water per dose of medicament to be initially stored in the inhaler over the expected “in-use” lifetime of the inhaler, typically taken to be 300 days. Preferably, the moisture penetration should be less than 0.01 mg of water per dose, and for some high-sensitivity medicaments should be less than 0.001 mg water per dose. During testing, and for comparison with the above figures, an appropriate driving force for moisture penetration (i.e. the water vapour transfer rate) would be an external Relative Humidity of 90%, an internal Relative Humidity of 0%, and a temperature of 38° Celsius. Such choice of materials and construction leads to a device that is effectively “hermetically sealed”.

Preferably, the inhaler is further characterised by the feature that the dose-storage region of the inhaler is raised internally to above atmospheric pressure. More preferably, an inert gas is used to raise the internal pressure to above atmospheric pressure.

Advantageously, the inhaler is a dry powder inhaler. Advantageously also, the dry powder is stored as an agglomerate or pellet, and the inhaler further comprises means for disrupting said pellet or agglomerate during its dose-dispensing cycle.

In any embodiment of the invention, there is advantageously provided a scavenger—such as desiccant or an oxygen scavenger—in gaseous communication with the dose-storage region of the inhaler.

The invention also provides a method of filling such inhalers, the method comprising the steps of charging the inhaler with a desired number of individually packaged doses; raising the pressure of the dose-storage region of the inhaler internally to above atmospheric pressure; and sealing the container in a manner which will resist depressurisation whilst allowing individual dose dispensation via the moisture-proof aperture.

### BRIEF DESCRIPTION OF THE DRAWINGS

The invention will be described with reference to the accompanying drawings in which:

FIG. 1 is a schematic diagram illustrating an arrangement of pre-determined doses of medicament within a moisture-proof enclosure.

FIG. 2 illustrates the operation of a moisture-proof aperture and dispensing means to convey medicament from within the inhaler to a dose-delivery position.

FIG. 3 illustrates the operation of a moisture-proof dose dispensing aperture through which individual doses of medicament may be moved from within a moisture-proof enclosure to an external, i.e. dose-delivery position.

### DESCRIPTION OF PREFERRED EMBODIMENTS

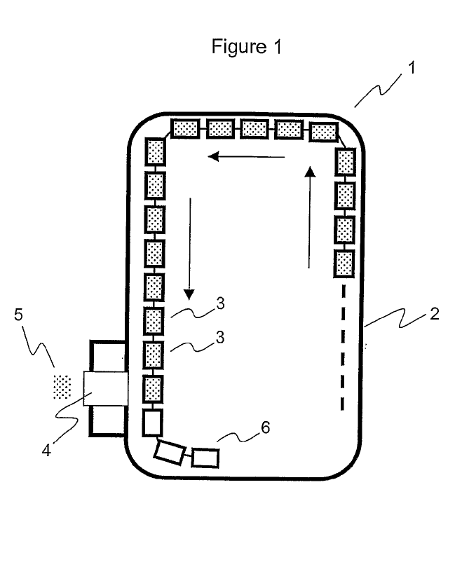

With reference to FIG. 1, in preferred embodiments of the invention, there is provided an inhaler generally indicated by 1, that comprises a moisture-proof barrier 2 that comprises substantially or partially an external region of the inhaler 1, and encloses or defines a chamber containing a plurality of individual doses of medicament 3 without enclosing the doses individually. The moisture-proof barrier 2 incorporates a moisture-proof aperture 4 through which the medicament doses 3 can, in use, be individually dispensed to a dose-delivery position 5. The individual doses within the inhaler can be indexed into position, in use, and as indicated schematically by the arrows in FIG. 1 to deliver successive doses of the medicament, and retaining empty dose-containing means 6 within the reservoir. Suitable means of indexing and presentation of individual doses to a dose-dispensing region are well known in the art and can be readily selected by one skilled in the art without further inventive thought.

### Embodiment 1

### Spool Valve

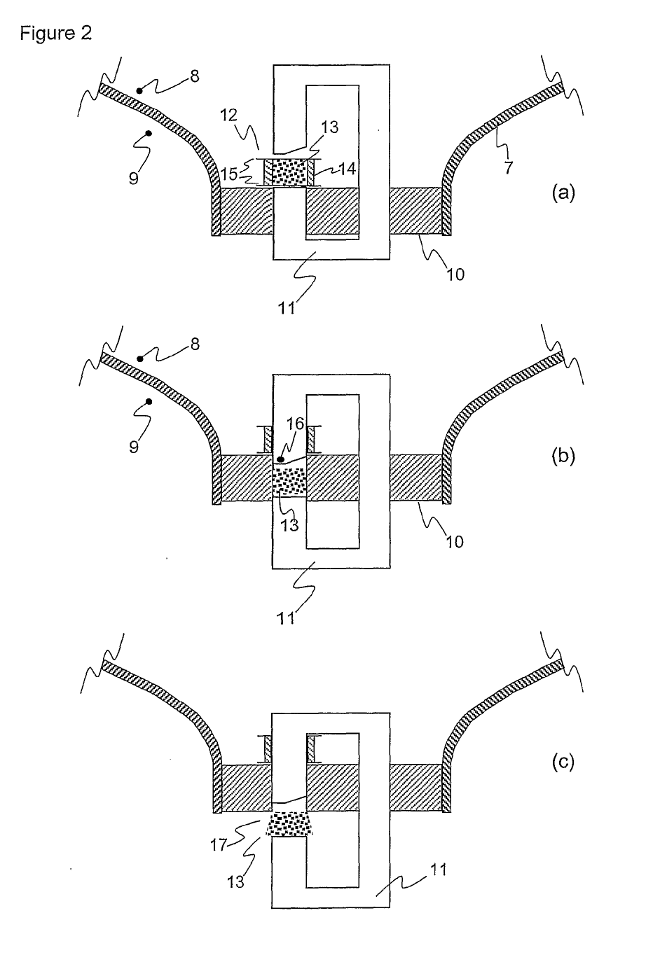

FIG. 2 illustrates a portion of an embodiment of the invention using a spool-type valve as the moisture-proof aperture. In this embodiment the moisture-proof barrier comprises a spun or deep-drawn aluminium canister, illustrated in part as 7, and which defines an interior chamber 8 and an exterior space 9. The canister 7 incorporates a moisture-proof aperture itself comprising a plug 10 made of a plastics material such as PTFE or a particularly moisture-proof plastics material such as COC (a cycloolefin co-polymer) and a generally C-shaped valve member 11 that passes through holes in the plug 10. The plug 10 could also conveniently and advantageously be made of a moisture proof material such as a metal or a glass. Individual pre-metered doses of medicament 12 are stored within the chamber 8. In FIG. 2a one such dose is illustrated as 12 in a position ready for dose-delivery. This medicament dose comprises the medicament itself 13, contained within a generally cylindrical member 14 made from an inexpensive material such as paper, card or plastics. Each end of the cylindrical member 14 is capped by a lid portion 15 that may easily be punctured to release the medicament 13. By virtue of the outer moisture-proof barrier, none of the containing means 14, 15 for the medicament 13 needs itself to be moisture-proof. They may therefore be manufactured cheaply and their design can be dictated by a wish to maximise the packing density of individual doses 12 within the canister rather than to achieve protection against moisture, oxygen and other degradative agents. Thus, the lid portion 15 may be formed from e.g. paper.

FIG. 2b illustrates this embodiment of the invention during a dose-delivery cycle. The valve member 11 is actuated to puncture the lid elements 15 of the medicament dose 12, thus transferring the medicament itself 13 to a space within the body of the plug 10. To facilitate this, one end 16 of the valve member 11 is shaped to facilitate rupture of the lid portions 15. The relative sizing of the valve member 11 and the plug 10 is such that during the transition from its closed position, illustrated in FIG. 2a to its dose-delivery position illustrated in FIG. 2c, there is never a path between the outside 9 and the inside 8 of the inhaler.

FIG. 2c illustrates this embodiment of the invention with the valve member 11 in its dose-delivery position. In this position, the medicament 13 has been moved from within the container 8 to an exterior position 17 where it may be inhaled by a user. In this embodiment, a small amount of air from the exterior space 9 may be transferred into the interior chamber 8 when the spool valve resets from the position shown in FIG. 2(c) to the position shown in FIG. 2(a). The volume of air thus transferred would, at most, be equivalent to the volume contained between the two faces of the C-shaped member 11, and within the aperture in the plug 10. If this volume is small, compared to the interior volume of the canister, then the small ingress of air is likely to be immaterial. If, however, the medicament is particularly sensitive to components in the outside air (e.g. water or oxygen) then raising the pressure inside the canister also has the effect of further diluting (i.e. on a mass or molar basis, rather than a volume basis) these components. Further advantages of pressurising the canister are discussed elsewhere.

### Embodiment 2

### Piston Valve

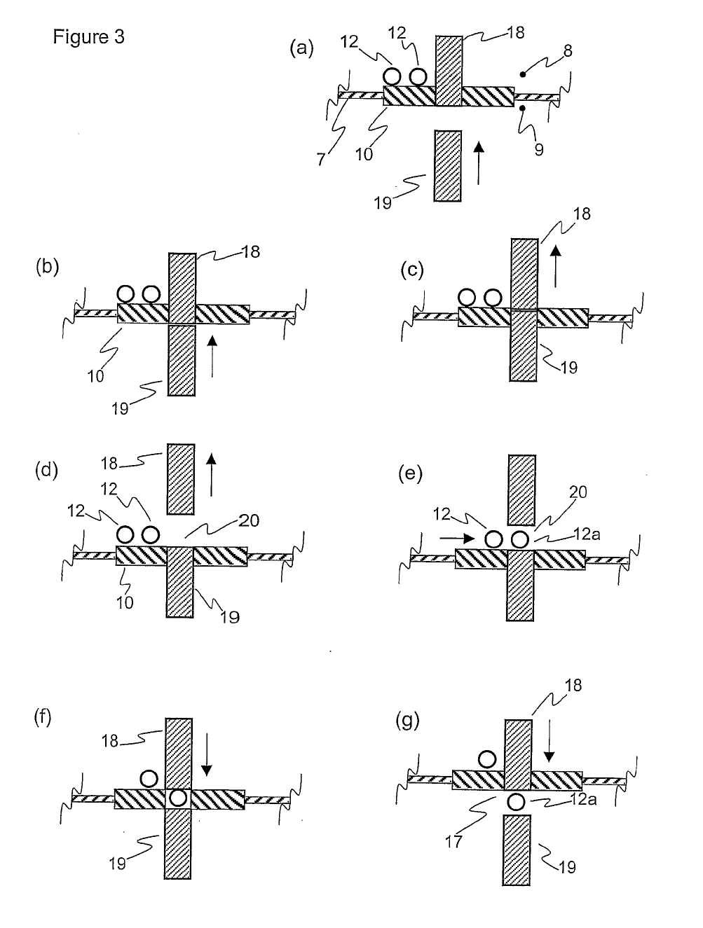

FIG. 3 illustrates a further embodiment of the invention. In this embodiment the moisture-proof barrier comprises a canister, shown in part as 7, which defines an interior space 8 and an exterior space 9 of the inhaler. This moisture-proof barrier incorporates a similarly moisture-proof aperture comprising a plug 10 and a pair of piston-like elements 18, 19 operating through a hole in the plug 10.

FIG. 3a illustrates this embodiment in a position ready to dispense a medicament dose. Two such doses 12 are illustrated for clarity but the interior space 8 would be capable of containing many more. A dose-dispensing cycle is illustrated in FIGS. 3a through to 3g. In FIG. 3b the piston element 19 is actuated towards the plug 10, engages with piston element 18 and, as illustrated in FIG. 3c, both elements 18 and 19 move towards the interior of the inhaler 8. In FIG. 3d, piston element 19 has stopped flush with the interior surface of plug 10, forming a moisture-proof seal. Element 18 continues along the same path creating a space 20 for receipt of a medicament dose 12. FIG. 3e illustrates the indexing of the medicament doses within the inhaler causing one of them, 12a, to move into the dose-dispensing space 20. FIG. 3f illustrates the movement of piston element 18 back towards the plug and sealing the aperture within it. In FIG. 3g, piston elements 18 and 19 both moved towards the exterior 19 of the inhaler, thus delivering the medicament dose 12a into a region 17 where it can be inhaled. The mechanism is now effectively re-set to a position identical to that in 3a, and thus ready for the delivery of the next dose.

In an embodiment such as this, further advantageous features may also be envisaged. In order to facilitate the actuation of the piston elements 18 and 19, a second aperture in the canister may be provided, through which piston element 19 passes. This has the benefit that actuation of both piston elements 18 and 19 may be effected from the exterior space 9. A benefit of this feature is that the actuation mechanism is separated from the medicament-containing region of the inhaler, and thus may be designed without the constraints of ingress protection required to protect the medicament from degradative agents.

A further advantageous feature that may be introduced concerns the relative motion of the two piston elements 18 and 19. Most dry-powder formulations designed for inhalation are stored in just such a dry powder form, often with a dry carrier, e.g. as a micronised (i.e. as particles or the order of a micron in diameter) pharmaceutical with somewhat larger lactose carrier particles. However, there are advantages to producing a loosely pelleted or agglomerated, formulation to facilitate the handling of doses. In cases such as this, the motion of the piston elements 18 and 19 may be readily be designed such that the individual pellet or agglomerate is broken up by the action of the pistons 18,19 during the dose-delivery cycle. Such an action could most effectively be achieved at the point in the dose-delivery cycle illustrated in FIG. 3(f). At this stage, the piston element 18 could move towards its position illustrated in FIG. 3(g) before piston element moves from its position in FIG. 3(f) to that in FIG. 3(g). This would create the crushing action needed to disrupt the pellet or agglomerate.

### Additional Medicament—Protecting Measures

In any embodiment of the invention, it is advantageous to raise the interior dose-containing and dose-dispensing region of the inhaler to above atmospheric pressure. This may be effected by the use of compressed, dry air. However, it may preferably be accomplished by the use of an inert gas. Suitable gases include nitrogen, argon and helium. This feature has a number of advantages. Firstly, the use of an inert gas prevents oxidative damage of the medicament doses contained within the inhaler. Secondly, an internal pressure above atmospheric pressure acts against the ingress of moisture, oxygen or other degredative agents during the lifetime of the medicament doses. Thirdly, the increased pressure within the inhaler will assist the dispensation of individual doses through the moisture-proof aperture. Fourthly, the increased pressure will assist in the creation of an aerosol dispersion of the medicament prior to inhalation. This is illustrated in FIG. 2 where a dry powder medicament 13 in positions 2a and 2b is surrounded by gas at the higher pressure corresponding to the interior region 8. When the valve element 11 reaches its dose-delivery position, illustrated in FIG. 2c, the expansion of the gas causes the medicament charge 13 to be expelled more rapidly, thus assisting the formation of an aerosol dispersion. Fifthly, the additional matter in the canister would serve to dilute and gases or vapours that may enter the canister.

Again, in any embodiment of the invention, a further degree of protection of the predicament from the degredative effects of oxygen and water may be obtained by including a scavenger in the dose-storing region of the inhaler. A desiccant may be used to scavenge water, and an oxygen-absorbing material (such as a readily-oxidised metal) may be used to scavenge oxygen. Whilst the use of such materials may have disadvantages in the reservoir-type of inhaler (see above), their use in the non-reservior type is less problematic. Firstly, as the medicament may be stored in dose-containing means such as illustrated in FIGS. 1 and 2, these means provide a physical separation of the medicament from the scavenger. Secondly, the provision of the moisture-proof barrier in the inhaler results in a decreased quantity of scavenger being required to maintain appropriate conditions for the medicament.

### Further Advantageous Features

In any embodiment of the invention, one or more further advantageous features are also envisaged:
A dose counter may be conveniently incorporated into the device. When the device is loaded with medicament doses, the counter would be set to the number of doses loaded. Then, as each dose is dispensed, the counter would track the reducing number of doses remaining. The number of remaining doses could then conveniently be displayed on the unit. Such a dose counter could also lock the device when no more doses remain, to alert the user, so preventing the user using the device in the mistaken belief that medicament is being delivered and with resultant “under-dosing”. Given this teaching, various options for the design and implementation of such a dose counter would be apparent to one skilled in the field of mechanical/electrical design.
Timing means, interacting with the dose dispensing mechanism, may also be provided, and optionally linked to data logging means. By suitable configuration, the timing means may then be used to control the frequency with which doses may be dispensed. Thus, a user may be prevented from “over-dosing” the medication by too-frequent use. Details of the timing of each dose may be stored by the data logging means. In this way, use of the medication may be tracked by the user, or by a supervising clinician. This would be particularly advantageous during clinical trials to check patient compliance with dosing regimes, or for clinicians to check the intensity of use of an “ad libitum” prescribed medication. Again, given this teaching, various methods of implementation would be apparent to the skilled addressee.
A typical dry powder formulation for administration by inhalation would comprise a micronised pharmaceutical together with a carrier, such as lactose, of a significantly larger particle size. In order to ensure delivery of the medicament to the desired location in the airway, it is essential that any agglomerates of these powders are broken up. The use of a pressurised canister is one way to aid this process (see above), but other modifications to break up agglomerates may also be incorporated, such as: the creation of turbulence in the device by the use of modified air paths; design of the airpath to encourage impaction of any agglomerates against a surface; the use of static electricity and the use of impaction devices such as propellers or impingers. Again, given this teaching, various methods of implementation would be apparent to the skilled addressee.
Whilst the dispensing of a dose may be actuated by e.g. a switch, it would be particularly advantageous to actuate the dose delivery by the breath of a user. In this way, the dispensing can be made at the most advantageous point of the inhalation cycle. Again, given this teaching, various methods of implementation would be apparent to the skilled addressee.
In order to reduce unwanted retention of medicament in the device, particularly in the dose-dispensing region, anti-static materials should be used in construction.
In order to increase hygiene, and to prevent foreign bodies entering the device, any mouthpiece would advantageously be provided with a cover.
In use, situations may arise where a user has dispensed a dose of medicament into a position where it may be inhaled, but inhalation of the dose does not follow. This may occur if the user is e.g. interrupted. There is a risk that the user may forget the already-dosed medicament, and attempt a second dose-cycle, so leading to “overdosing” of the medicament. In order to prevent this, an additional feature may be introduced to clear any earlier dosed medicament from the “inhalation zone” prior to dosing. A simple mechanical device could achieve this. Alternatively, a jet of gas could conveniently be delivered from a gas reservoir to automatically clear the “inhalation zone” prior to dosing. If this gas was the same inert gas used to pressurise the container, the gas jet could also be arranged to flush air from in and/or around the moisture-proof valve to reduce the issue of air ingress discussed above. Again, given this teaching, various methods of implementation would be apparent to the skilled addressee.
Actuation of the dose dispensing mechanism could advantageously be allied to the operation of and lid of the device (e.g. a mouthpiece cover).
For pediatric use, it is preferable to use a reservoir between inhalation devices and the user; these are known in the art as “spacers”. Such a spacer could conveniently and advantageously be incorporated into a device of the present invention.
To encourage patient compliance—especially for the young or fashion conscious—particular advantage would be gained by providing the device with means for “customisation” of its exterior shape, colour and/or texture to meet the desires and aspirations or the intended user.

It will be apparent to the skilled addressee that a device according to the present invention would be suitable for a wide range of inhaled medicament formulations such as drug-lactose blends, modified particles and drug co-formulations.

1. An inhaler of the non-reservoir kind (i.e. one which operates by dispensing individually packaged doses) characterised by the feature that a moisture-proof barrier—which may optionally comprise substantially or partly an external region of the inhaler—encloses the dose-storage region of the inhaler without enclosing the doses individually, and incorporates a similarly moisture-proof aperture through which the medicament doses can, in. use, be individually dispensed.

2. An inhaler according to claim 1 characterised by the feature that the dose-storage region of the inhaler is raised internally to above atmospheric pressure.

3. An inhaler according to claim 2 wherein an inert gas is used to raise the internal pressure to above atmospheric pressure.

4. An inhaler according to claim 1 wherein the inhaler is a dry powder inhaler.

5. An inhaler according to claim 4 wherein the dry powder is stored as an agglomerate or pellet, and the inhaler further comprises means for disrupting said pellet or agglomerate during its dose-dispensing cycle.

6. An inhaler according to claim 1 further comprising a scavenger—such as desiccant or an oxygen scavenger—in gaseous communication with the dose-storage region of the inhaler.

7. A method of filling an inhaler according to claim 2, the method comprising the steps of charging the inhaler with a desired number of individually packaged doses; raising the pressure of the dose-storage region of the inhaler internally to above atmospheric pressure; and sealing the container in a manner which will resist depressurisation whilst allowing individual dose dispensation via the moisture-proof aperture.
